(19) United States
(12) Patent Application Publication
(10) Pub. No.: US 2011/0004133 A1
(43) Pub. Date: Jan. 6, 2011

(54) BRACE WITH TARGETED COOLING

(75) Inventors: Victor Viner, Northfield, IL (US); Walter Brown Herbst, Lake Forest, IL (US); Anthony Pannozzo, Needham, MA (US); Gregory Kenny, Wakefield, MA (US); Rose Heath, Melrose, MA (US); John Depiano, Burlington, MA (US); Noel Leon Gauthier, Norwood, OH (US)

Correspondence Address: Klintworth & Rozenblat IP LLC, 300 West Adams St., Suite 423, Chicago, IL, 60606, US

(73) Assignee: V2 Capital, LLC., Glenview, IL (US)

(21) Appl. No.: 12/520,070

(22) PCT Filed: Dec. 19, 2007
(86) PCT No.: PCT/US07/88182

§ 371 Date: Jul. 22, 2010

Related U.S. Application Data
(60) Provisional application No. 60/875,610, filed on Dec. 19, 2006.
(60) Provisional application No. 60/875,868, filed on Dec. 20, 2006.
(60) Provisional application No. 60/885,036, filed on Jan. 16, 2007.

(51) Int. Cl. A61F 5/01

(52) U.S. Cl. 602/14; 602/26

(57) ABSTRACT

A brace providing targeted cooling to predetermined anatomical locations and further providing controlled compression. The brace can be wrapped and fastened around a body part. The brace having an interior layer providing comfort to a users skin and capable of efficient and controlled thermal conductivity, and having an outer layer providing insulation and controlled compression. The brace further including a cooling bladder with cooling nodules providing both targeted therapeutic cooling when the brace is worn.

### CROSS-REFERENCES TO RELATED APPLICATIONS

The Present Application claims priority to U.S. Provisional Patent Application Nos. 60/875,610, filed 19 Dec. 2006; 60/875,868, filed 20 Dec. 2006; and 60/885,036, filed 16 Jan. 2007. The contents of each of these U.S. Provisional patent applications are hereby incorporated herein in their entireties.

### FIELD OF THE PRESENT INVENTION

The Present Invention relates generally to therapeutic and first aid devices, and more particularly, to therapeutic and first aid devices having a cooling system targeting anatomical points for efficient treatment.

### BACKGROUND OF THE PRESENT INVENTION

It is customary for ailing body parts to be treated with the “R.I.C.E.” system of therapy. The R.I.C.E. therapy system comprises Rest, Ice, Compression and Elevation. The “Ice” portion of the therapy calls for cooling the injured anatomy, which serves to improve healing and the treatment of aches, pains and/or inflammation. Various methods and substances have been used to achieve the anatomic cooling. These methods and substances range from applying topical solutions with penetrating cooling effects, to applying ice packs and/or cold compresses against the injured area. However, it is inefficient and dangerous to apply cooling substances directly to a general anatomic area because it limits the user's mobility during the cooling therapy and, more importantly, increases the danger of hypothermic conditions (which may include, for example, congelatio, or “frostbite”). Further, where the cooling is achieved by the application of ice packs and/or cold compresses, maintaining a large surface area of the cold pack in contact with a general anatomic area causes the ice to melt, which has the negative consequence of raising the temperature of the cold compress.

A variety of cold packs currently known in the art provide for anatomical cooling. However, they do not protect the user from the dangers of hypothermic conditions and/or over cooling. Further, currently-known cold packs do not integrate protection from condensation (that is, currently-known cold packs additionally suffer from the naturally-occurring phenomenon of condensation as a result of the heating—due to either body temperature or the ambient air around the cold pack—of the cooling substance). Thus, there remains a need for a product that allows users to efficiently cool body parts without concern of either hypothermic conditions and/or condensation.

The “Compression” portion of the R.I.C.E. therapy system calls for constriction of the injured area, thereby treating any current inflammation and reducing future inflammation. The constriction also provides support to the injured area, allowing for normal (or, more appropriately, near normal) movement. However, it is both inefficient and dangerous to have uncontrolled constriction, which can occur with the utilization of air casts, elastic wraps and/or elastic braces without professional supervision. For example, uncontrolled constriction may act as a tourniquet of sorts, cutting off blood flow to various parts of the body “downstream” from the injured area. Further, uncontrolled constriction can restrict range of movement in both the injured area and in other proximately-located parts of the body, which impedes the healing process and prolongs “stiffness” of the area. Thus, there exists a need for a device which provides only the adequate amount of constriction of the injured area, without requiring professional supervision for utilization.

Finally, it is desirable to complete the R.I.C.E. therapy system in an efficient manner. It is inefficient to have separate materials for the “Ice” portion of the therapy and for the “Compression” portion of the therapy. Consequently, it is desirable to complete the “Ice” portion of the therapy with an efficient cooling device, and it is desirable to complete the “Compression” portion of the therapy with a device providing controlled constriction without professional supervision.

Thus, a need exists to overcome the disadvantages listed herein, and to provide a device capable of performing multiple therapeutic functions efficiently.

### SUMMARY OF THE PRESENT INVENTION

The Present Invention provides a device, such as a brace, for supporting and treating a body part. In one embodiment, the device preferably includes an inner layer configured to contact a body part and provide controlled thermal conductivity. Further, the device preferably includes a cooling bladder connected with the inner layer and configured to be in thermal connection with at least one anatomical point, wherein the cooling bladder defines at least one cooling nodule. Preferably, the cooling nodule targets cooling to at least one predetermined anatomical point for therapeutic purposes. The device also preferably includes an outer layer, connected to the cooling bladder, having a first end and a second end, and configured to substantially hold the inner layer against a body part. The device also preferably includes a fastening means connected with the first end of the outer layer and configured to substantially join the first and second ends of the outer layer. Finally, the device preferably includes an adjusting means connected with the outer layer, configured to adjust the outer layer for fit to a body part.

In another embodiment, the Present Invention preferably provides for a device for supporting and treating a body part, such as a knee. The device preferably includes an inner layer that may be placed in contact with the skin around the knee and may provide thermal conductivity, allowing a cooling effect to pass through to the knee without undesired interference. Further, the device may preferably include a cooling bladder connected to the inner layer and configured to pass cool temperatures to an anatomical point around the knee with a cooling nodule, intended to target the passing of cool temperatures to a predetermined anatomical point for therapeutic purposes. Finally, the device may preferably include an outer layer with a first boundary and a second boundary, wherein the outer layer is connected to the cooling bladder and configured to hold the inner layer in contact with the skin around the knee.

In a typical implementation the cooling bladder may include substances which reduce the freezing point of water (such as propylene glycol, and/or a thickening agent), allowing the bladder to be in a semi-solid state during temperatures ranging from below freezing to above body temperature. The cooling bladder may be cooled by placing the device in a freezer or similar apparatus. The volume capacity of the cooling bladder may be minimized by having an outer layer capable of insulating the cooling bladder from the ambient temperatures. The outer layer may include neoprene, POLARTEC®, THINSULATE™, SUPPLEX®, CORDURA® and/or similar thin insulating material which allows moisture to be wicked away.

In a typical implementation of the device, the cooling bladder may be configured to efficiently pass cooling effects to the knee, for example, both when the knee is flexed and when the knee is extended. Further, the device may include a fastening means, which may be connected to a first boundary of the outer layer and may allow the joining of the first and second boundaries. Such fastening means may include a hook and loop fastener (e.g., velcro), hooks, zippers, spandex, clips, clasps, buckles and other similar fasteners. In this way, the device may be wrapped and fastened around the body part.

In further implementations of the device, the device may include a supplemental cooling bladder connected to the fastening means, allowing cooling substantially around the periphery of the body part. Further, the device may include an adjusting means disposed on or about the outer layer, which preferably allows adjustment of the outer layer and/or the device to fit the body part. Like the fastening means discussed above, such adjusting means may include a hook and loop fastener (e.g., velcro), hooks, zippers, spandex, clips, clasps, buckles and other similar fasteners.

In another aspect, the Present Invention provides for a device for supporting and treating a body part, such as a knee. Preferably, the device includes an inner layer placed in contact with the skin of the wearer to providing controlled thermal conductivity. The device preferably also includes a cooling bladder connected to the inner layer and having at least one cooling nodule, wherein the cooling nodule is configured to be in thermal connection with at least one predetermined anatomical point for therapeutic purposes. The device preferably also includes at least one air bladder connected to the cooling bladder and configured to substantially hold a gaseous substance. Finally, the device preferably includes an outer layer having a first and a second boundary. In operation, the outer layer may connect to the air bladder and hold the inner layer against the skin of the body part.

In one implementation, the device may include a plurality of air bladders. In another implementation, the device may include an air pump configured to inflate the air bladder. In another implementation, the device may include an air release valve configured to deflate the air bladder. In another implementation, the air bladder may be configured to hold the cooling bladder against the user such that the cooling effects of the cooling bladder continue both while the body part is flexed and/or extended.

Further, the device, in this implementation, may include a fastening means, which may be connected to a first boundary of the outer layer and may allow the joining of the first and second boundaries. Such fastening means may include a hook and loop fastener (e.g., velcro), hooks, zippers, spandex, clips, clasps, buckles and other similar fasteners. In this way, the device may be wrapped and fastened around the body part.

In further implementations of the device, the device may include a supplemental cooling bladder connected to the fastening means, allowing cooling substantially around the periphery of the body part. Further, the device may include an adjusting means disposed on or about the outer layer, which preferably allows adjustment of the outer layer and/or the device to fit the body part. Like the fastening means discussed above, such adjusting means may include a hook and loop fastener (e.g., velcro), hooks, zippers, spandex, clips, clasps, buckles and other similar fasteners.

In another aspect, the Present Invention provides for a device for supporting and treating a body part. Preferably, the device includes an inner layer configured to contact the skin of a body part and provide controlled thermal conductivity. The device preferably also includes a cooling bladder connected to the inner layer and configured to be in thermal communication with at least one anatomical point of the body part, wherein the cooling bladder defines at least one cooling nodule, and wherein the cooling nodule targets cooling to the at least one anatomical point for therapeutic purposes. The device preferably also includes an insulating layer connected to the cooling bladder configured to insulate the cooling bladder. The device preferably also includes a compression layer having a first and a second end in active communication with the insulating layer and configured to substantially hold the inner layer in substantial contact with the body part. The device preferably also includes at least one fastening means in releasable communication with the compression layer and configured to substantially join the first and second ends of the compression layer. Finally, the device preferably includes an outer layer connected to the compression layer.

In an implementation of the device, the fastening means of the device may include a supplemental cooling bladder allowing cooling substantially around the periphery of the body part.

In another aspect, the Present Invention provides for a kit for a carrying a brace and maintaining the cooling capabilities of the brace for an extended period of time. The kit preferably includes a case configured to be carried and provide insulation and a brace configured to cool and support a body part.

An advantage of the Present Invention is that the cooling nodules may maintain contact with the targeted anatomical areas while the body is in motion, providing targeted cooling and controlled compression. Further, the inner layer may sufficiently control the cooling effects of the cooling bladder such that the wearer may immediately use the device upon removing it from a freezer or similar apparatus without concern of hypothermic conditions. Similarly, the inner and outer layers may sufficiently control the effects of condensation caused by the cooling bladder. These advantages allow the wearer to efficiently receive cooling therapy and compression therapy from a single device, without requiring professional supervision for effective utilization.

Other objects, advantages and capabilities of the Present Invention will become apparent from the following Detailed Description Of The Present Invention, taken in conjunction with the accompanying Figures showing the various embodiments of the Present Invention.

### BRIEF DESCRIPTION OF THE FIGURES

The Present Invention will best be understood with reference to the following Detailed Description Of The Present Invention, taken in conjunction with the accompanying Figures, in which:

FIG. 1 illustrates a side view of a brace manufactured in accordance with the tenets and teachings of the Present Invention;

FIG. 2 illustrates an exploded perspective view of a cooling bladders and an air bladders used in one embodiment of the brace of FIG. 1;

FIG. 3 illustrates a front view of the cooling bladder of the brace of FIG. 1;

FIG. 4 illustrates a top view of the cooling bladder of the brace of FIG. 1;

FIG. 5 illustrates a rear view of the cooling bladder of the brace of FIG. 1;

FIG. 6 illustrates a side view of the cooling bladder of the brace of FIG. 1;

FIG. 7 illustrates an isometric view of the cooling bladder of the brace of FIG. 1; and

FIG. 8 illustrates an exploded view of a brace manufactured in accordance with the tenets and teachings of the Present Invention.

### DETAILED DESCRIPTION OF THE PRESENT INVENTION

Referring to the Figures, wherein like Reference Numerals are used to identify like components, FIG. 1 illustrates a side view of brace 10 as applied to a body part (in the Figures, the example of the body part illustrated is a knee), and illustrating outer layer 12 and air pump 20. Preferably, air pump 20 allows the user or wearer of brace 10 to inflate an air bladder (not visible in FIG. 1). Although the embodiment illustrated in FIG. 1 is illustrated as applicable to a knee, it is nevertheless contemplated that the Present Invention may be developed to be applicable to other injured body parts as well, and the Present Invention should not be construed as being only applicable to the knee.

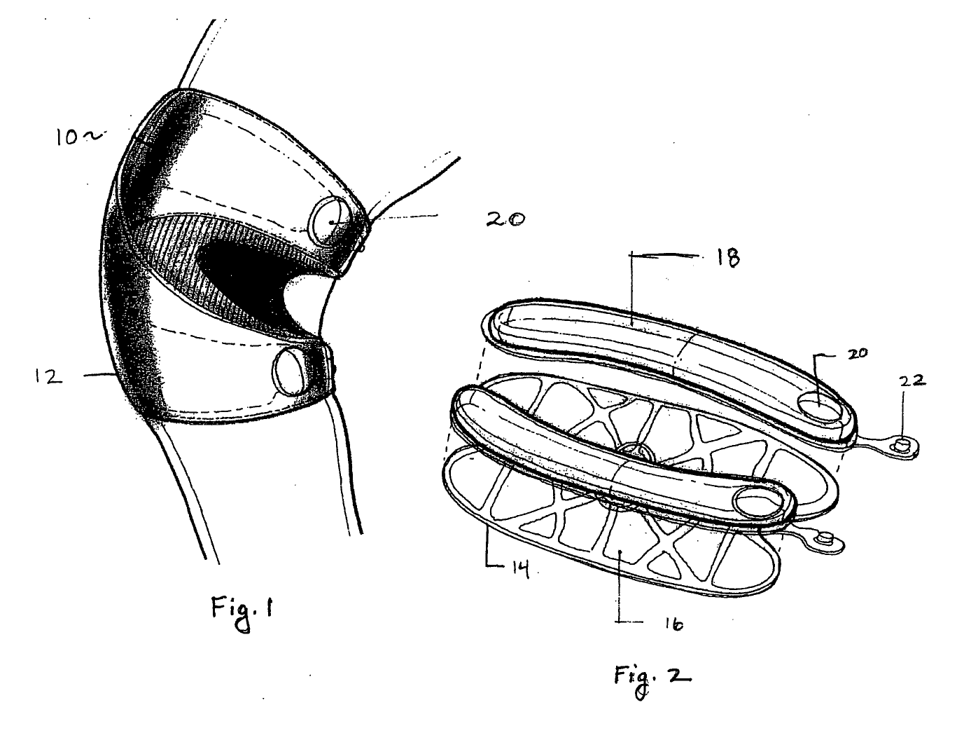

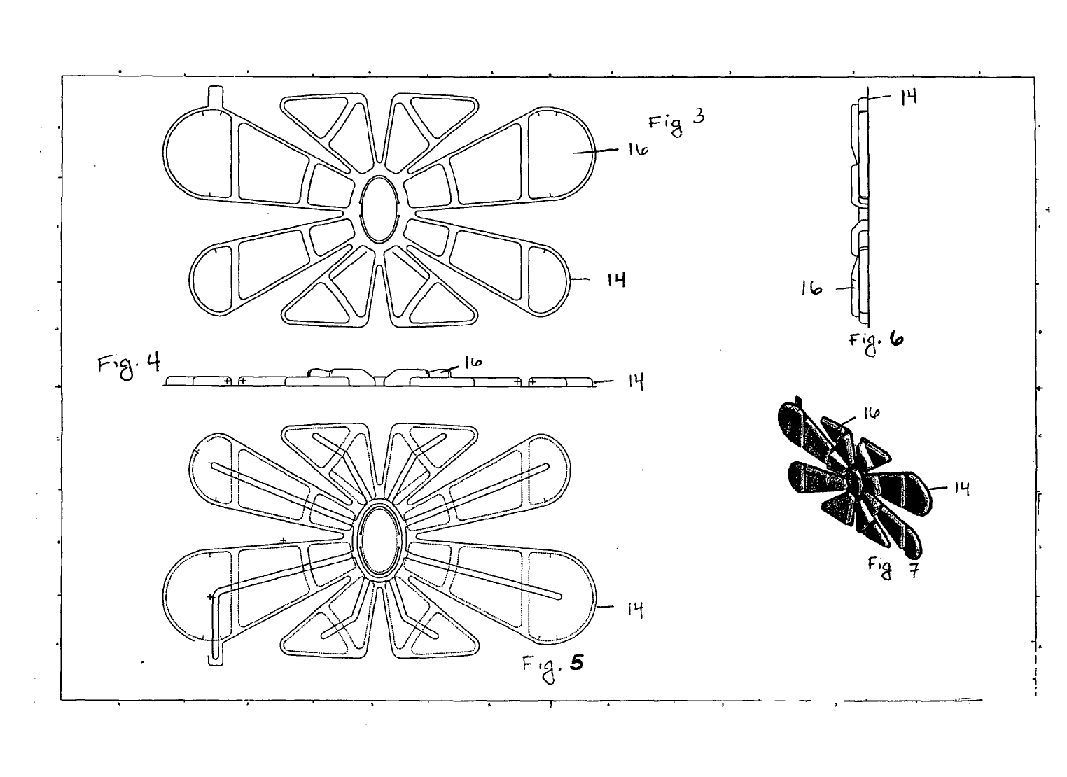

The interior of brace 10, illustrated in FIG. 2, includes cooling bladder 14, which is used to provide targeted cooling to anatomical points around a body part. Cooling bladder 14, also illustrated in FIGS. 3-7, provides targeted cooling to various predetermined anatomical points of the wearer through strategically-located or strategically-disposed cooling nodules 16 placed at appropriate locations in relation to the anatomical point to be cooled. In doing so, cooling bladder 14 can preferably be configured to be in thermal connection with at least one anatomical point of the body part intended to be cooled.

Further, cooling bladder 14 may be configured to provide cooling to anatomical points around substantially the entire periphery of the body part, while allowing the wearer freedom of movement of that body part. For example, the cooling bladder 14 can be configured to provide strategic cooling superior and inferior to the patella of the wearer.

It is preferred that cooling bladder 14 comprise a durable material that will minimize leaking caused by excessive temperature exposure, extreme pressure, inadvertent puncturing or any other possible condition which may compromise cooling bladder 14. It is also preferred that the composition of both cooling bladder 14 and cooling nodules 16 include substances which reduce the freezing point of water (such as propylene glycol or a thickening agent), allowing cooling bladder 14 and cooling nodules 16 to be in a semi-solid state during temperatures ranging from below freezing to above body temperature.

Cooling bladder 14 and cooling nodules 16 may be cooled by placing the entire of brace 10 (or, alternatively, a portion thereof) in a freezer or similar temperature-controlled cooling apparatus. The volume capacity of cooling bladder 14 and cooling nodules 16 may be minimized by configuring outer layer 12 to be capable of insulating cooling bladder 14 and cooling nodules 16 from the ambient temperature. Preferably, outer layer 12 may include neoprene, POLARTEC®, THINSULATE™, SUPPLEX®, CORDURA® and/or similar thin insulating material which allows moisture to be wicked away. Finally, it is preferred that cooling bladder 14 and cooling nodules 16 comprise a material sufficient to achieve an ideal cooling profile for a predetermined period of time—such as when brace 10 is outside of the temperature-controlled cooling apparatus, while still being able to maintain a soft and pliable foundation, allowing brace 10 to articulate properly around the body part.

The predetermined targeted anatomical points to be cooled may include the quadriceps tendon, the medial tendon, the lateral patellar retinaculum, the patella, the patellar tendon, the hamstring tendon, medial ligaments, lateral collateral ligaments and any other similar anatomical points which surround anatomical joints. Alternatively, the Present Invention may be used to cool any aspect of the human body. Further, cooling bladder 14 and cooling nodules 16 may be configured—together or separately—to contact substantially around the periphery of, for example, a knee above a patella, and may pass cooling effects around a quadriceps tendon. In this implementation, cooling bladder 14 and cooling nodules 16 may also be configured to contact substantially around the periphery of the knee below a patella, and may pass cooling effects around a patellar tendon. Finally, cooling bladder 14 and cooling nodules 16 may be configured to efficiently pass cooling effects to the knee, both when the knee is flexed and when the knee is extended.

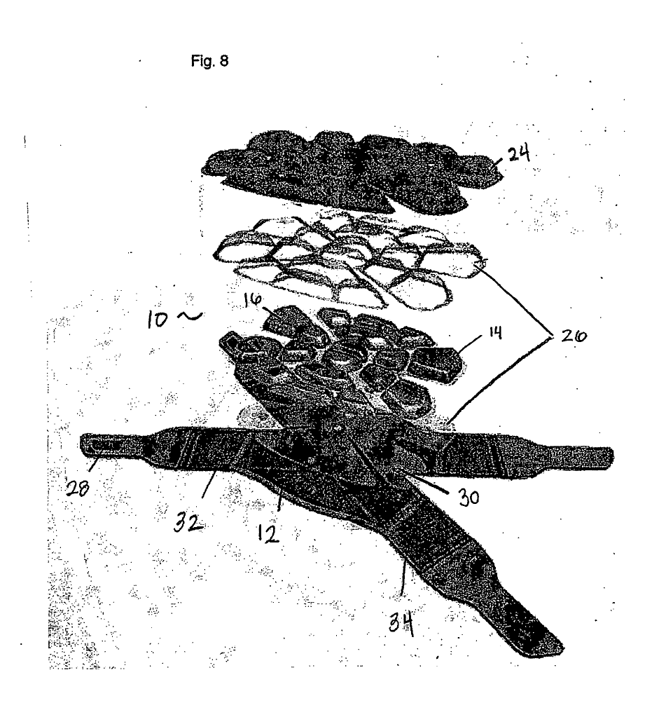

Returning to FIG. 1, and as further illustrated in FIG. 8, it is preferred that brace 10 also include inner layer 24. Inner layer 24 is preferably configured to be in substantial contact with the body part of the wearer. In doing so, inner layer 24 can provide controlled thermal conductivity to the body part. In this way, inner layer 24, by preventing the direct application of cooling bladder 14 and/or cooling nodules 16 to the body part, proscribes the possibility of hypothermic conditions that may be realized by the wearer. Thus, in this manner, cooling bladder 14 is in active communication with inner layer 24, and not directly with the body part of the wearer.

It is preferable that inner layer 24 of brace 10 provides sufficient insulation and conductivity so as to protect the wearer from hypothermic conditions when the cooling bladder is at or near freezing temperatures, yet will allow the skin to maintain a temperature range of approximately Forty-Eight-Seventy Degrees Fahrenheit (48-70° F.) after consistent application of the device for approximately Twenty (20) Minutes. The inner layer preferably comprises a material having the same or similar properties as DRI-LEX®; that being, a material that is lightweight, quick-drying, breathable and odor- and mildew-resistant, and is able to provide a degree of climate control to the wearer. Further, the inner layer may preferably provide additional protection to wearer. For example, the inner layer may include an antimicrobial substance to protect the user from hazardous microbes.

Further, it is preferred that both inner layer 24, cooling bladder 14 and cooling nodules 16 be contained within outer layer 12. Outer layer 12 acts as an encasement device for brace 10, and acts to substantially maintain both inner layer 24, cooling bladder 14 and cooling nodules 16 in substantial contact with the body part.

Although not necessary, it is further preferred that brace 10 be a “wrap” device; that is, brace 10 is preferably configured to be attached to the body part in such a fashion that it requires a fastening means to be secured thereto. This is in contrast to a “sleeve” device, in which a device is tubular in shape and slips on the body part. In a “wrap” device, brace 10 is wrapped around the body part and a fastening means is used to secure brace 10 to the body part. Further, brace 10 may comprise at least one supplemental cooling bladder 14, which may be located on a fastening means, to allow the cooling effects of brace 10 to reach the periphery of a body part, such as the posterior of the knee.

Further, bearing in mind that not all body parts are of the same size, it is preferred that brace 10 have at least one adjusting means on or about outer layer 12, which may allow adjusting of outer layer 12 and/or brace 10 to fit the body part. Such adjusting means may comprise a hook and loop fastener system (e.g., velcro), loops, latches, ratchets, clasps and buckles, a zipper and/or lacing. Preferably, the adjusting means is in active communication with outer layer 12 of brace 10, and allows the wearer to adjust the fit of brace 10.

Additionally, brace 10 may include a fastening means, which may be connected to a first boundary of outer layer 12 and may allow for the joining of the first and second boundaries. In operation and in conjunction with the fastening means, brace 10 may be wrapped and fastened around a body part. Like the adjustment means, described above, the fastening means may comprise a hook and loop fastener system (e.g., velcro), loops, latches, ratchets, clasps and buckles, a zipper and/or lacing.

As illustrated in FIG. 2, the interior of brace 10 includes at least one air bladder 18. Air bladder 18 is preferably located between outer layer 12 (not visible in FIG. 2) and cooling bladder 14. It is preferred that air bladder 18 be configured to hold a gaseous substance, such as air. Preferably, air bladder 18 is in fluid communication with air pump 20 and air release valve 22. Air pump 20 inflates air bladder 18 to adjust the compression provided by brace 10. Air release valve 22 deflates air bladder 18 to adjust the compression provided by brace 10. Air bladder 18 is preferably intended to operate very similar to a blood pressure measurement device, in that, upon the insertion of air into the bladder, it provides for a compression of brace 10 upon the body part. Thus, it is intended that the wearer, once brace 10 is secured onto the body part, can utilize air pump 20 and air release valve 22 to provide a comfortable compression level of brace 10. Thereby, the wearer can adjust the compression provided by brace 10 for comfort, support and therapeutic purposes.

Reference is now made to FIG. 8, which illustrates an exploded view of another embodiment of the Present Invention. The embodiment illustrated in FIG. 8 includes insulating layer 26 in communication with cooling bladder 14 in such a circumscribing manner so as to allow for insulation of cooling bladder 14, and preventing the escape or evaporation of the contents of cooling bladder 14. Preferably, insulating layer 26 may be composed of material that stays soft and pliable when cooling bladder 14 is maintained at temperatures below freezing. That is, insulating layer 26 is preferably designed to cushion the body part while, at the same time, maintaining an ideal cooling profile for the wearer. Preferably, the material is further designed to provide a barrier to evaporation, thereby extending the life of brace 10. Insulating layer 26 may also be releasably connected to outer layer 12 by at least one connector 30. Connector 30 may be a magnet, snap or any other known similarly releasable mechanism.

Further it is preferred that outer layer 12 include first end 32 and second end 34 for attachment to the body of the wearer. Additionally, outer later 12 is intended to be configured to substantially hold inner layer 24 in substantial contact with the body part. This can be accomplished by, for example, having mesh, elastic or other similar aspects included in outer layer 12, which exert a pressure upon brace 10 such that it fits snugly to the body of the wearer. It is preferred, in this embodiment, that at least one fastening means 28 be in releasable communication with outer layer 12 and configured to substantially join first and second ends 32, 34 of outer layer 12, allowing for connection and disconnection between the various elements of brace 10. A preferred fastening means for this embodiment is a strap with clasps, where the strap with clasps includes a supplemental cooling bladder. However, as set forth above concerning the other fastening and adjusting means, the fastening means in this embodiment may comprise a hook and loop fastener system (e.g., velcro), loops, latches, ratchets, clasps and buckles, a zipper and/or lacing.

A further embodiment of the Present Invention comprises a kit for transporting a portable brace 10. Preferably, the kit would include a case (or other similar device) for carrying and insulating brace 10, and brace 10, as described above. The case described herein may preferably comprise any known type of portable carrying mechanism in which brace 10 may be disposed, and may also be configured to substantially maintain the temperature of cooling bladder 14 and cooling nodules 16. The case may comprise molded plastic, cloth or any other similar material that, in addition to providing a carrying means, would also be durable enough to shield brace 10 from the elements and shock caused as a result of dropping.

While only those embodiments set forth above have been described in detail, other configurations and embodiments for the Present Invention may nevertheless exist that are within the spirit and scope of the Present Invention.

What is claimed is:

1. A device for supporting and treating a body part comprising:
an inner layer configured to substantially contact the body part and provide controlled thermal conductivity;
a cooling bladder in active communication with the inner layer and configured to be in thermal communication with at least one anatomical point of the body part, the cooling bladder defining at least one cooling nodule, wherein the cooling nodule targets cooling to the at least one anatomical point for therapeutic purposes;
an outer layer having a first end and a second end, the outer layer in active communication with the cooling bladder and configured to substantially hold the inner layer in substantial contact with the body part;
at least one fastening means in active communication with the first end of the outer layer and configured to substantially join the first and second ends; and
at least one adjusting means in active communication with the outer layer configured to adjust the outer layer for fit to the body part.

2. The device of claim 1, further including an air bladder in active communication with the outer layer and the cooling bladder configured to substantially hold a gaseous substance.

3. A device for supporting and treating a knee comprising:
an inner layer configured to contact the knee and provide controlled thermal conductivity;
a cooling bladder in active communication with the inner layer and configured to be in thermal communication with at least one anatomical point around the knee, the cooling bladder defining at least one cooling nodule, wherein the at least one cooling nodule targets cooling to the at least one anatomical point for therapeutic purposes; and
an outer layer having a first boundary and a second boundary, the outer layer in active communication with the cooling bladder and configured to substantially hold the inner layer in active communication with the knee.

4. The device of claim 3, wherein the cooling bladder is configured to contact substantially around the periphery of the knee superior to a patella and in substantial thermal connection with a quadriceps tendon, and the cooling bladder is configured to contact substantially around the periphery of the knee inferior to the patella and in substantial thermal connection with a patellar tendon, medial tendon, lateral patellar retinaculum, patella, patellar tendon, hamstring tendon and quadricep tendon.

5. The device of claim 3, wherein the cooling bladder is configured to maintain substantial thermal connection with the knee during flexion and extension.

6. The device of claim 3, further including at least one fastening means in active communication with the first boundary of the outer layer and configured to substantially join the first and second boundaries.

7. The device of claim 6, further including a supplemental cooling bladder in active communication with the at least one fastening means configured to cool substantially around the periphery of the knee.

8. The device of claim 3, further including at least one adjusting means in active communication with the outer layer configured to adjust the outer layer to fit the knee.

9. A device for supporting and treating the knee comprising:
an inner layer configured to contact the knee and provide controlled thermal conductivity;
a cooling bladder in active communication with the inner layer and defining at least one cooling nodule, wherein the at least one cooling nodule is configured to be in thermal communication with at least one anatomical point of the knee for therapeutic purposes;
at least one air bladder in active communication with the cooling bladder and configured to substantially hold a gaseous substance; and
an outer layer having a first and a second boundary, the outer layer in active communication with the at least one air bladder and configured to substantially hold the inner layer in contact with the knee.

10. The device of claim 9, further including a plurality of air bladders.

11. The device of claim 9, further including an air pump configured to inflate the at least one air bladder.

12. The device of claim 9, further including an air release valve configured to deflate the at least one air bladder.

13. The device of claim 9, wherein the at least one air bladder is configured to maintain the cooling bladder in substantial thermal communication with the knee during flexion and extension of the knee.

14. The device of claim 9, further including at least one fastening means in active communication with the first boundary of the outer layer and configured to substantially join the first and the second boundaries.

15. The device of claim 14, further including a supplemental cooling bladder in active communication with the fastening means configured to cool substantially around the periphery of the knee.

16. The device of claim 9, further including at least one adjusting means in active communication with the outer layer configured to adjust the outer layer to fit the knee.

17. A device for supporting and treating a knee comprising:
an inner layer configured to contact the knee and provide controlled thermal conductivity;
a cooling bladder in active communication with the inner layer and configured to be in thermal communication with at least one anatomical point around the knee, wherein the cooling bladder is configured to contact substantially around the periphery of the knee superior to a patella and in substantial thermal connection with a quadriceps tendon, and the cooling bladder is configured to contact substantially around the periphery of the knee inferior to a patella and in substantial thermal connection with a patellar tendon, and the cooling bladder is configured to maintain substantial thermal connection with the knee during flexion and extension;
the cooling bladder defining at least one cooling nodule, wherein the cooling nodule targets cooling to at least one anatomical point for therapeutic purposes;
an outer layer having a first boundary and a second boundary, the outer layer in active communication with the cooling bladder and configured to substantially hold the inner layer in active communication with the knee;
at least one fastening means in active communication with the first boundary of the outer layer and configured to substantially join the first and second boundaries;
a supplemental cooling bladder in active communication with the fastening means configured to cool substantially around the periphery of the knee; and
at least one adjusting means in active communication with the outer layer configured to adjust the outer layer to fit the knee.

18. The device of claim 17, further including at least one air bladder in active communication with the cooling bladder and configured to substantially hold a gaseous substance.

19. A device for supporting and treating a body part comprising:
an inner layer configured to substantially contact the body part and provide controlled thermal conductivity;
a cooling bladder in active communication with the inner layer and configured to be in thermal communication with at least one anatomical point of the body part, the cooling bladder defining at least one cooling nodule, wherein the cooling nodule targets cooling to the at least one anatomical point for therapeutic purposes;
an insulating layer in active communication with the cooling bladder configured to insulate the cooling bladder;
a compression layer having a first and a second end in active communication with the insulating layer and configured to substantially hold the inner layer in substantial contact with the body part;
at least one fastening means in releasable communication with the compression layer and configured to substantially join the first and second ends of the compression layer;
an outer layer in active communication with the compression layer.

20. The device of claim 19, wherein the fastening means includes a supplemental cooling bladder configured substantially cool around a body part periphery.

21. A kit comprising:
a case configured to be carried and provide insulation; and
a brace configured to cool and support a body part;
wherein the kit maintains the cooling capabilities of the brace for an extended period of time.
